(19) United States
(12) Patent Application Publication
(10) Pub. No.: US 2005/0033207 A1
(43) Pub. Date: Feb. 10, 2005

(54) Immobilizing apparatus, and methods of use and manufacture

(75) Inventor: Robert Anders, Scottsdale, AZ (US)

Correspondence Address: Schwegman, Lundberg, Woessner & Kluth, P.A., P.O. Box 2938, Minneapolis, MN, 55402, US

(73) Assignee: 3FI Products LLC

(21) Appl. No.: 10/897,840

(22) Filed: Jul. 23, 2004

Related U.S. Application Data
(60) Provisional application No. 60/492,354, filed on Aug. 4, 2003.

(51) Int. Cl. A61F 5/00

(52) U.S. Cl. 602/8

(57) ABSTRACT

An apparatus functions to immobilize a body part or other physical object. In an embodiment, the apparatus includes a base, one or more flexible bags, and one or more foam precursor packages. In an embodiment, a foam precursor package includes a first chamber, in which a first foam precursor is deployed, and a second chamber, in which a second foam precursor is deployed. The foam precursor package also includes a separation mechanism, which when physically agitated, functions to enable the first and second foam precursors to combine. The combination produces a reaction, which results in the formation of a foam. The foam substantially fills the one or more flexible bags. In an embodiment, a portion of a patient's body is placed in proximity to the apparatus, prior to combination of the precursors. When the foam precursor package is activated, the foam and flexible bags contour around and at least partially immobilize the body part.

### CROSS-REFERENCE TO RELATED APPLICATION

This application claims benefit to provisional application No. 60/492,354, filed on Aug. 4, 2003.

### TECHNICAL FIELD

The inventive subject matter relates generally to foam-in place apparatus, and more specifically to foam-in-place apparatus adapted to immobilize portions of the body or other physical objects, and methods of making and using such apparatus.

### BACKGROUND

Emergency medical technicians (E.M.T.) are often called to accident scenes in which a person has sustained a bodily injury that should be immobilized. For example, when a patient has a potential head, neck or spinal cord injury, an E.M.T. performs roughly the following procedure. First, the E.M.T. applies a cervical collar, which is a device that is wrapped around the patient's neck to keep the patient's neck midline. Next, the patient is transferred onto a rigid spine board. This is typically performed by rolling the patient on one side, placing the board under the patient, then rolling the patient back onto the board. At this point, the E.M.T. places a “head immobilizer” around the patient's head and cervical collar to prevent any side-to-side motion of the patient's head or neck during transport to an emergency medical facility. If such motion occurs, permanent physical damage could result.

A head immobilizer typically is a cardboard or plastic device, which needs to be unfolded, assembled, and then taped to the spine board in order to be effective. Current head immobilizers are cumbersome devices, which are difficult for the E.M.T. to work with, particularly while the E.M.T. is wearing his or her protective gloves. More importantly, they are extremely time-consuming to use, which places the patient at even more risk of permanent damage or death.

Another problem that exists with current head immobilizers is that, while taping the head immobilizer to the spine board, the E.M.T. usually is forced to reach underneath the board. Often, this results in the E.M.T.'s protective gloves becoming torn on glass or other obstacles or debris located under the board. In addition, because of the difficulty of handling tape with the protective gloves on, and because of the risk of exacerbated injury if the process of securing the head immobilizer takes too long, the E.M.T. may remove his or her gloves during the process. Obviously, torn or removed protective gloves place the E.M.T. at a much greater risk of being exposed to blood or other bodily fluids. Yet another problem with current methods of injury immobilization is that, the more tape that is used in order to immobilize the patient, the more time it will take hospital personnel to remove the head immobilizer, so that they can fully care for the injury.

Techniques for immobilizing other areas of the body (e.g., joints, hands, feet, limbs, shoulders, etc.) also suffer from problems similar to the problems encountered with head and neck immobilization. In addition, medical professionals are sometimes forced to manipulate injured body parts to apply current immobilization devices. This manipulation can further injure the patient.

Besides the medical industry, other industries also continue to develop ways of immobilizing objects in a manner that the objects are less likely to become damaged. For example, a wide variety of packaging technologies have been developed to secure objects being shipped in shipping containers. Often, these packaging technologies require the use of extensive external equipment, which limits their use to companies that have sufficient manufacturing facilities. These technologies are not portable, and thus are generally unavailable to the average person.

### BRIEF DESCRIPTION OF THE DRAWINGS

FIG. 1 illustrates a top-down, front view of an immobilizing device adapted to immobilize a portion of the body, in accordance with an embodiment;

FIG. 2 illustrates a top-down, front view of an immobilizing device, in accordance with an alternate embodiment;

FIG. 3 illustrates a top-down, front view of an immobilizing device, in accordance with another alternate embodiment;

FIG. 4 illustrates a top-down, front view of a reactant container, in accordance with an embodiment;

FIG. 5 illustrates a bottom-up, back view of the immobilizing device of FIG. 1, in accordance with an embodiment;

FIG. 6 illustrates a flowchart of a method for using an immobilizing device, in accordance with an embodiment;

FIG. 7 illustrates a first perspective view of an immobilizing device, prior to activation, in a folded position within a pre-heating storage unit, in accordance with an embodiment;

FIG. 8 illustrates a second perspective view of an immobilizing device, prior to activation, in a folded position, in accordance with an embodiment;

FIG. 9 illustrates a perspective front view of an immobilizing device, prior to activation, in an unfolded position, in accordance with an embodiment;

FIG. 10 illustrates a perspective back view of an immobilizing device, prior to activation, with a portion of an adhesive protection sheet removed, in accordance with an embodiment;

FIG. 11 illustrates a perspective view of an immobilizing device, prior to activation, affixed to a support structure, in accordance with an embodiment;

FIG. 12 illustrates a perspective view of the immobilizing device of FIG. 11, prior to activation, with a patient positioned on the device, in accordance with an embodiment;

FIG. 13 illustrates a perspective top view of the immobilizing device and patient of FIG. 12, prior to activation, in accordance with an embodiment;

FIG. 14 illustrates a cross-sectional, head-down view of an immobilizing device and body part, in accordance with an embodiment;

FIG. 15 illustrates a cross-sectional, head-down view of an immobilizing device and body part, in accordance with another embodiment;

FIG. 16 illustrates a perspective view of the immobilizing device and patient of FIG. 12, after activation, in accordance with an embodiment;

FIG. 17 illustrates a side, cross-sectional view of a flexible receptacle, prior to activation, in accordance with an embodiment;

FIG. 18 illustrates a side, cross-sectional view of a flexible receptacle, during activation, in accordance with an embodiment;

FIG. 19 illustrates a side, cross-sectional view of a flexible receptacle, in a fully expanded position after activation, in accordance with an embodiment;

FIG. 20 illustrates a flowchart of a method for manufacturing an immobilizing device, in accordance with an embodiment;

FIG. 21 illustrates a perspective view of an immobilizing device adapted to immobilize an arm, in accordance with an embodiment;

FIG. 22 illustrates a perspective view of an immobilizing device adapted to immobilize a leg, in accordance with another embodiment; and

FIG. 23 illustrates a perspective, cut-away view of two immobilization devices within a shipping container, in accordance with an embodiment.

### DETAILED DESCRIPTION

Various embodiments of the inventive subject matter, described in detail herein, involve new and novel methods and apparatus adapted to immobilize a body part or other physical structure, and a method of manufacturing the apparatus. Embodiments of the invention have several significant advantages over prior art methods.

First, various embodiments of the apparatus are easy for E.M.T. to use to immobilize a body part while the E.M.T. is wearing protective gloves. Further various embodiments of the apparatus can immobilize a body part more completely and quickly than the head immobilizer system that has been used in the past. Further, various embodiments of the apparatus can immobilize a body part completely, while still being quick and easy to remove.

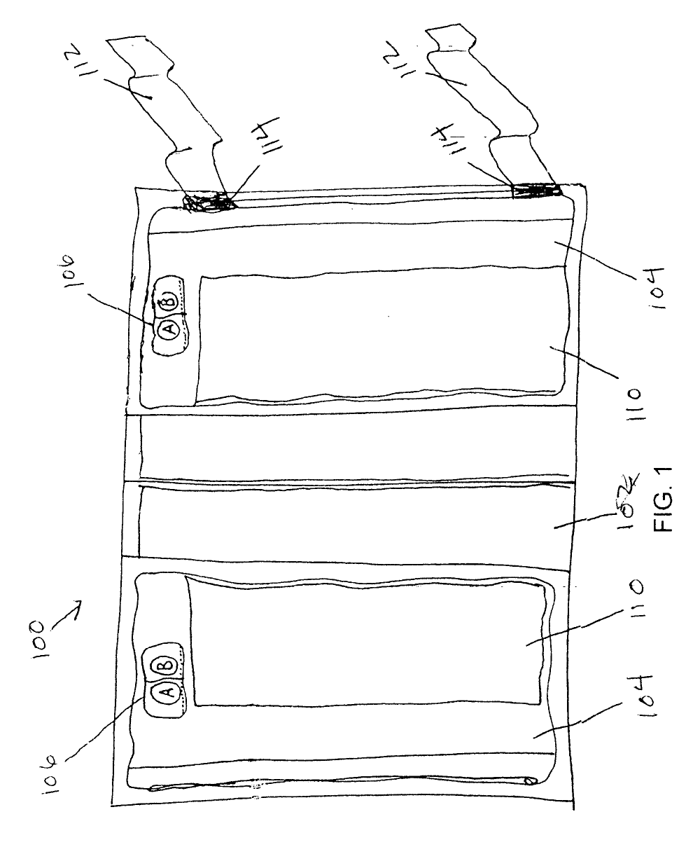

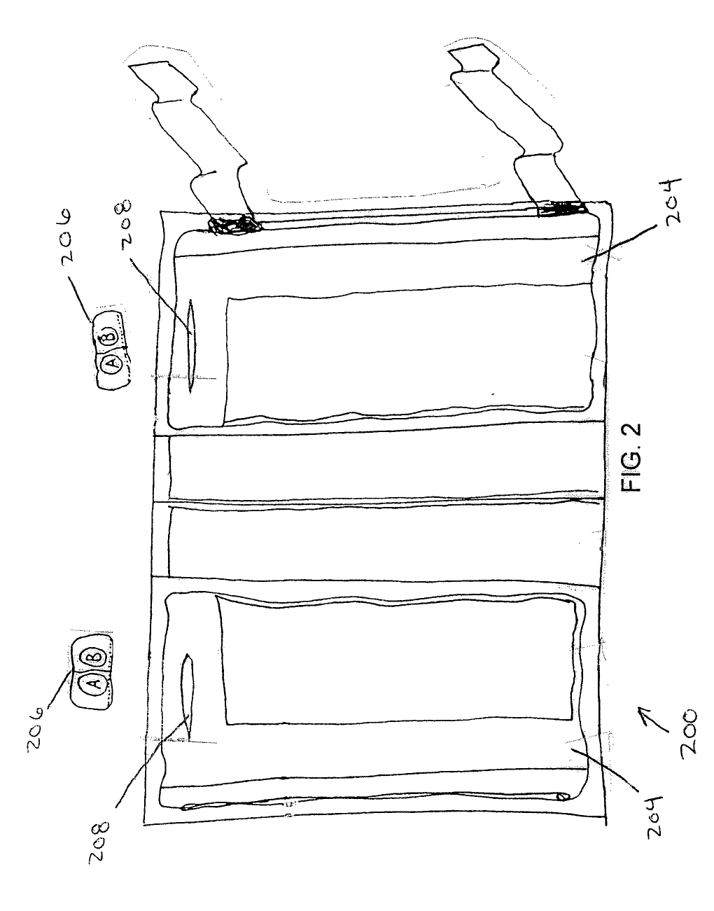

FIG. 1 illustrates a top-down, front view of an immobilizing device 100 adapted to immobilize a portion of the body, in accordance with an embodiment. The device 100 includes a base 102, one or more flexible bags 104, and one or more foam precursor packages 106. In an embodiment, the device 100 also includes an adhesive surface on the back of the base (illustrated in FIG. 2), one or more insulator layers 110, and one or more securing straps 112 and fasteners 114.

The base 102 can be formed from a rigid, semi-rigid or flexible material. In an embodiment, the base 102 is formed from cardboard, which allows the device 100 to retain its shape, and also facilitates properly positioning the device on a spine board or other support structure. The size of the base 102 depends on the size of the body part it is intended to immobilize, and the size of the surface that the base 102 is to be secured to. For example, when the device is designed to be secured to a spine board in order to immobilize a head, spine or neck, the base has have a width of approximately 18 inches, and a height of approximately 15 inches, in an embodiment, although the base can be wider, narrower, shorter, or taller, as well.

In an embodiment, the device 100 includes two flexible bags 104, each of which include an interior cavity into which immobilizing foam is deployable. The interior cavity is defined by an inside surface of the flexible bag 104. In an embodiment, bags 104 can be positioned on either side of a patient's head when the device 100 is secured to a spine board, and the patient is laid upon the spine board. In other embodiments, the device 100 includes only one bag, or includes more than two bags.

In other embodiments, the base 102 and bags 104 are relatively positioned in a manner that they can function to effectively immobilize other body parts. In still other embodiments, the base 102 and bags 104 are pre-attached or attachable to a rigid or semi rigid support structure other than a spine board. The support structure can be substantially flat, or can be contoured or molded in a manner that it loosely conforms to a body part to be immobilized. In still another embodiment, a splint is provided that does not include an attached base. Instead, in an embodiment, the flexible bag has significant rigidity to provide support for the foam as it is forming.

Flexible bags 104 are formed from plastic or any other flexible material that is capable of substantially retaining the immobilizing foam during activation of the device. In an embodiment bags 104 are airtight. In another embodiment, bags 104 are not airtight, but are nonetheless capable of substantially retaining the foam. For example, bags 104 include one or more perforated or vented portions (not shown), in an embodiment, which enable gaseous by-products to be vented from the bags 104 during formation of the foam.

In an embodiment, the one or more flexible bags are shaped to at least partially contour to a body part intended to be at least partially immobilized. In other words, in an embodiment, the one or more flexible bags have dimensions that are proportional to the dimensions of a body part intended to be at least partially immobilized. For example, but not by way of limitation, if the splint is intended to immobilize an entire leg, at least one of the flexible bags would have a length proportional to the length of a leg. Alternatively, if the splint is intended to immobilize a finger, the length of at least one of the flexible bags would be substantially shorter.

Flexible bags 104 are attached to base 102 using an adhesive, in an embodiment. In another embodiment, flexible bags 104 include areas that are attachable using a penetrating securing device (e.g., a nail, rivet, screw, staple, hook, or other device). Desirably, these areas do not extend into the cavity into which the immobilizing foam is deployed. In still other embodiments, flexible bags 104 are attached to base 102 using a non-permanent securing device, such as a hook-and-loop (e.g., Velcro) type fastener, a snap, hook, clip, button, or other such device.

In an embodiment, a foam precursor package 106, also referred to herein as a “reactant container,” is positioned within each flexible bag 104 during manufacture of the device 100. Desirably, the foam precursor package 106 is attached to a portion of the bag, in order to stabilize the precursor package and to make sure that it is located in a predictable position. The predictable position can be indicated by a marking (e.g., a circle or other shape) on the top surface of the flexible bag 104, to make it easy for the user to identify the location of the foam precursor package 106.

FIG. 2 illustrates a top-down, front view of an immobilizing device 200, in accordance with an alternate embodiment. In this embodiment, the foam precursor package is not positioned within the flexible bag during manufacture. Instead, as illustrated in FIG. 2, the flexible bag 204 includes an opening 208 for inserting the foam precursor package 206. Desirably, the opening 208 for inserting the package 206 is sealable, and does not allow the foam to escape the flexible bag 204 during activation of the device. For example, but not by way of limitation, the opening 208 can include a hook-and-loop type fastener, a temporary or permanent adhesive, a tongue-and-groove type fastener, or another sealable fastener.

FIG. 3 illustrates a top-down, front view of an immobilizing device 300, in accordance with another alternate embodiment. In this embodiment, the foam is not produced in the flexible bags by activation of a foam precursor package. Instead, as illustrated in FIG. 3, a valve or other opening 308 exists within the flexible bag 304 to allow a foam and/or reactant chemicals to be introduced into the interior cavity of the bag 304.

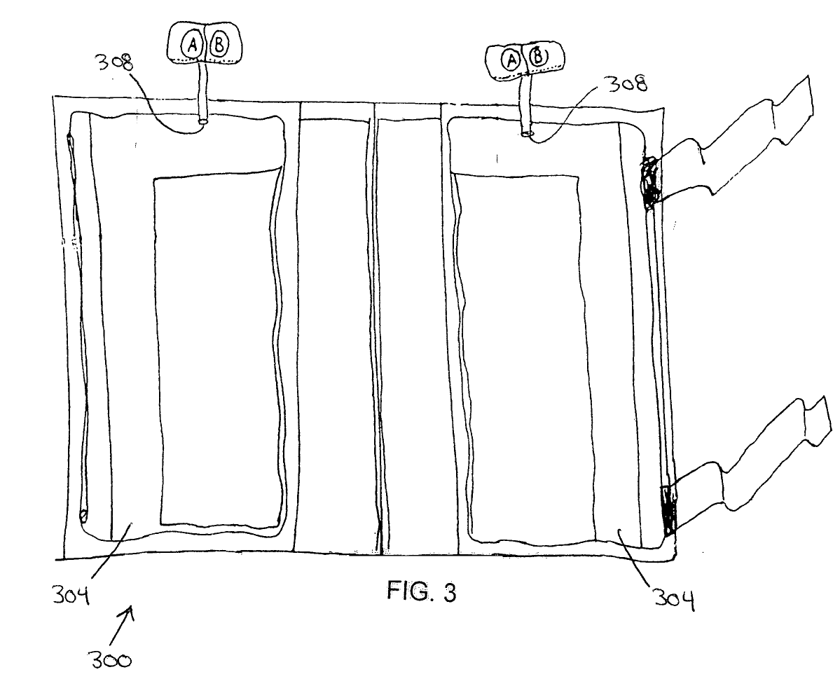

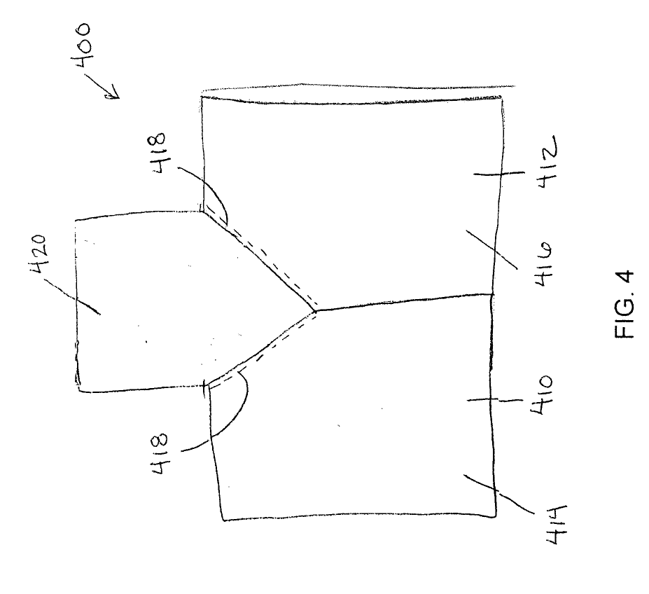

FIG. 4 illustrates a top-down, front view of a foam precursor package 400 (e.g., reactant containers 106, 206, 306, FIGS. 1-3), in accordance with an embodiment. Foam precursor package 400 includes a first chamber 414 and a second chamber 416, which function to contain and keep separate a first foam precursor 410 and a second foam precursor 412, also referred to herein as “reactant chemicals.” The first foam precursor 410 is disposed in the first chamber 414, and the second foam precursor 412 is disposed in the second chamber 416.

In addition, foam precursor package 400 includes one or more separation mechanisms 418, in an embodiment, which separate first and second chambers 414, 416 from a mixing chamber 420. Separation mechanism 418 can be a perforation, frangible barrier or other separation mechanism. Separation mechanism 418 can be readily broken or deflected. Accordingly, when the foam precursor package 400 is physically agitated or “activated,” the separation mechanism 418 functions to enable the first and second foam precursors 410, 412 to at least partially exit the first and second chambers 414, 416, respectively, and to combine within mixing chamber 420.

When the foam precursors 410, 412 are combined, they function to produce a reaction, which results in formation of a foam. During the reaction, the foam expands to fill the interior cavity of the flexible bag or bags (e.g., bags 104, 204, 304, 2104, 2204, (FIGS. 1-3, 21, 22)). Accordingly, the flexible bag functions to substantially contain the foam within its interior cavity during the reaction.

In an embodiment, the foam precursors 410, 412 include two chemicals, where each chemical is held in separation from the other chemical within the foam precursor package 400, prior to activation. When these two chemicals are pre-heated and combined, they expand and produce a foam, which rapidly sets and solidifies.

Polyurethane foam is produced from the combination of the first chemical 410 and the second chemical 412, in one embodiment. In other embodiments, the polyurethane foam is produced from the combination of more than two foam precursor components.

The first chemical 410 includes a member of the isocyanate family with a functionality in a range of 2.0-2.9 (e.g., a diisocyanate), including but not limited to a polymeric MDI, in one embodiment. The second chemical includes a polyol, in one embodiment, including but not limited to a polyether polyol. In another embodiment, the second precursor component includes a polyurethane foam resin.

Various additives may be included in the second chemical, in one embodiment. In alternate embodiments, additives are included in the first precursor component or in both the first and second precursor components. In one embodiment, additives are selected from a group of additives that includes a blowing agent precursor, a surfactant (i.e., a cell control agent), a catalyst, a cross-linker, and a flame retardant. In other embodiments, more, fewer, or additional additives can be included with either or both the first and second chemicals.

The term “Part A” is used herein to refer to the first chemical (e.g., including a diisocyanate) and any additives mixed with the first chemical. The term “Part B” is used herein to refer to the second chemical (e.g., including a polyol) and any additives mixed with the second chemical. In one embodiment, additives are mixed only with the second chemical. In other embodiments, additives are mixed with both chemicals. In still other embodiments, a portion of the polyol may be “pre-reacted” with the isocyanate component to form a “prepolymer” as Part A, which may then be reacted with the Part B component during device activation. In still another embodiment, a “Part C” component may be present, which may include, for example, a catalyst, a flame retardant, and/or another combination of additives. The Part C component may be held in separation from the Part A and Part B components until the device is activated.

When Part A and Part B are combined, various blowing agent precursors combine to generate a chemical blowing agent, which aids in the production of the foam. In one embodiment, the blowing agent is carbon dioxide (CO₂). The CO₂ may be generated, for example, by the reaction of diisocyanate in Part A with water (H₂O) as a blowing agent precursor in Part B, in one embodiment. In alternate embodiments, other chemical blowing agents can be generated through the reaction of Part A and Part B. Alternative physical blowing agents include, but are not limited to, chlorinated fluorocarbons (CFC), hydro chlorofluorocarbons (HCFC), hydro fluorocarbons (HFC), and hydrocarbons such as pentane or butane, for example, but not by way of limitation.

In other embodiments, other chemicals are used as the foam precursors, and/or one or more than two chemicals are used. Desirably, the foam precursors have a property that, when combined, they produce a foam that can readily fill the flexible bag (e.g., bag 104, 204, 304, 2104, 2204, (FIGS. 1-3, 21, 22)) in a manner that the foam and the bag conform to the patient's body. In addition, it is desirable that the foam produced by the foam precursors take a relatively short amount of time to solidify. For example, in an embodiment, the foam produced by the foam precursors substantially solidifies in less than 10 seconds. In another embodiment, the foam substantially solidifies within a time range of 10-30 seconds. In still another embodiment, the foam substantially solidifies in greater than 30 seconds. The foam precursors chosen to produce the foam may or may not need to be pre-heated prior to activation.

In an embodiment, a foam precursor package is formed from two flexible sheets that are selectively sealed to form the first and second chambers. In various other embodiments, a foam precursor package may be formed from one flexible sheet (e.g., folded over and selectively sealed), one or more substantially rigid substrates, a syringe device (e.g., a multiple chamber syringe), a substantially rigid canister, and/or one or more other materials or components.

Referring back to FIG. 1, in an embodiment, the device 100 also includes one or more insulator layers 110. These insulator layers 110 are desirably formed from a material that provides heat insulation. For example, the insulator layers 110 can be formed from a flexible foam, cloth, polymer, plastic, or other suitable material. An exothermic reaction can be produced when the foam precursors are combined. Accordingly, the insulator layers 110 function to insulate and protect the body part or other physical object from the heat associated with the exothermic reaction.

The layers 110 are attached to the base 102 and/or bags 104 in such a manner that, when activation occurs, the insulator layers 110 are disposed between the flexible bags 104 and the patient's body part or the other physical object. In another embodiment, the flexible bags 104 are formed from a material and/or have a thickness that provides the desired thermal protection, without the use of insulator layers 110.

In an embodiment, the device 100 also includes one or more securing straps 112 and fasteners 114. The securing straps 112 and fasteners 114 are stabilization mechanisms, which perform the function of holding the patient still in relation to the flexible bags 104 and foam, before, during, and/or after activation. For example, if the device is used to immobilize a patient's head, the flexible bags 104 and the foam within them provide a snug nest in which the patient's head rests. One securing strap 112 can be secured across the patient's forehead, and another securing strap 112 can be secured across the patient's chin. Both straps 112 can be removably secured to fasteners 114, so that the patient's head is held securely within the nest provided by the flexible bags 104 and foam.

In an embodiment, a first end of a strap 112 is either permanently or removably attached to a fastener 114 on a first region of flexible bag 104 or base 102, and a second end of the strap 112 is removably attached to another fastener 114 on a second region of flexible bag 104 or base 102. In an embodiment, the attachment between strap 112 and fastener 114 include a hook-and-loop type of attachment mechanisms. Alternatively, the attachment is made using a snap, hook, clasp, frictional, or other type of attachment mechanisms. In still other embodiments, types of stabilization mechanisms other than straps are used.

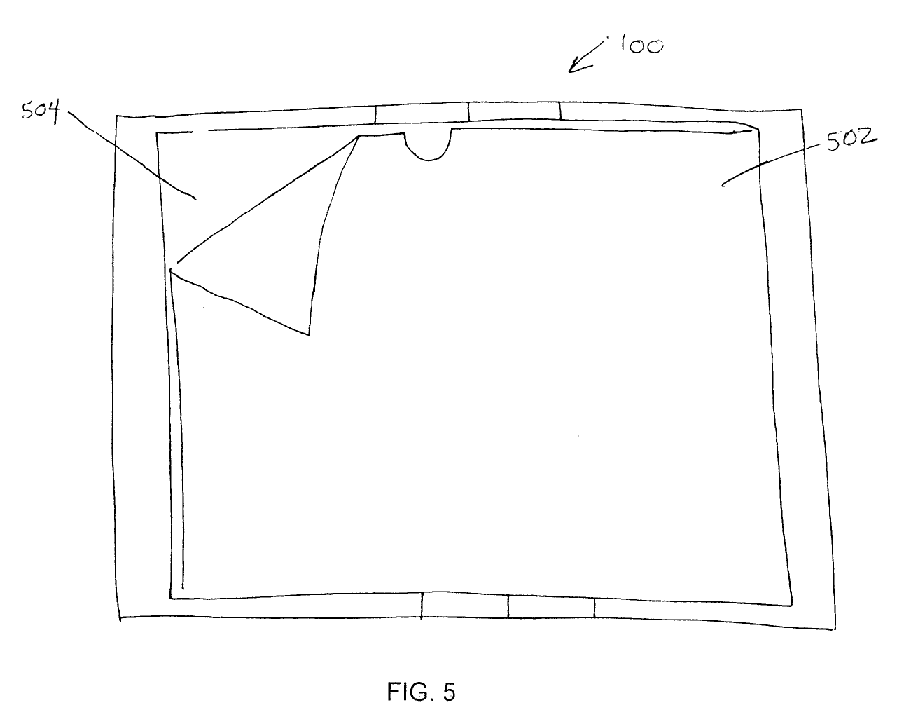

In an embodiment, the device 100 includes an adhesive surface on the back side of the base 102. FIG. 5 illustrates a bottom-up, back view of the immobilizing device 100 of FIG. 1, in accordance with an embodiment. The device includes an area that is covered by a removable protective sheet 502, which is shown partially pulled away in FIG. 5. Behind the protective sheet 502 is an adhesive surface 504. Once the protective sheet 502 is pulled away, the adhesive surface 504 is used to secure the device to a support structure, such as a spine board or other rigid or semi-rigid surface. In an embodiment, the material used for the adhesive surface 504 is sufficient to firmly attach the device 100 to a surface, but also allows the device 100 to be later removed from the surface without undue difficulty. In alternate embodiments, the device 100 is pre-attached or permanently attachable to the spine board or other support structure.

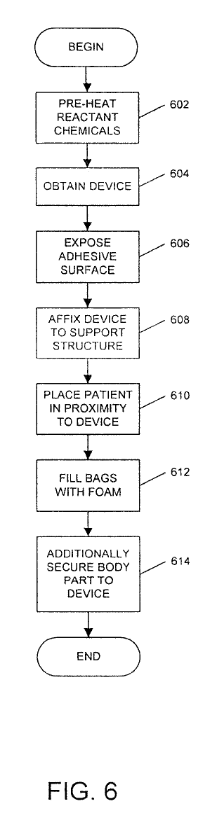

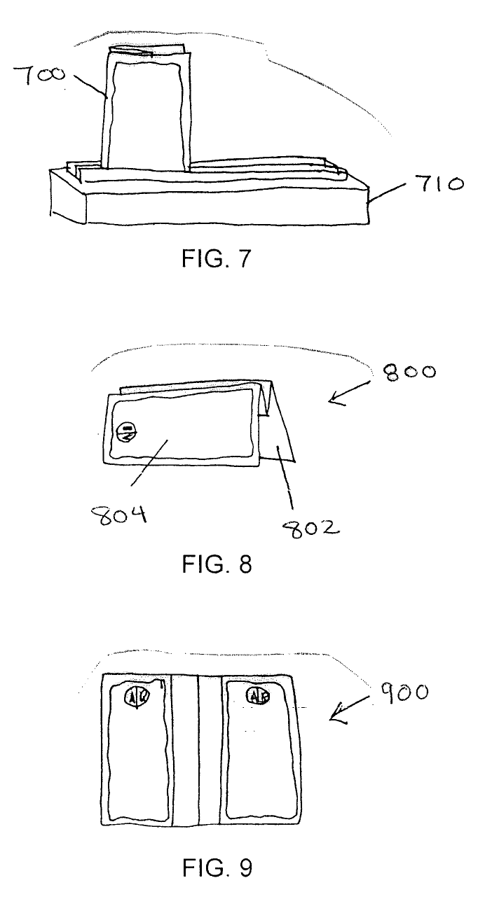

FIG. 6 illustrates a flowchart of a method for using an immobilizing device, in accordance with an embodiment. FIG. 6 should be viewed in conjunction with FIGS. 7-19, which illustrate various stages of using and activating an immobilizing device.

The method begins, in block 602, by pre-heating the foam precursors to a temperature sufficient to ensure that the desired reaction will occur when the chemicals are combined. In an embodiment, the foam precursor package is integrated with the device (e.g., located within the flexible bags) prior to use. In this embodiment, the entire device can be pre-heated, or the portion of the device that includes the foam precursor package can be inserted into a heating unit.

FIG. 7 illustrates a first perspective view of an immobilizing device 700, prior to activation, in a folded position within a pre-heating storage unit 710, in accordance with an embodiment. Pre-heating storage unit 710 includes slots, which are adapted to receive at least an end of the device 700, in a folded position, in an embodiment. Within the slots are heating elements (not shown), which are sufficient to produce a quantity of heat that will heat the foam precursor package to at least the minimum temperature required for a sufficient reaction between the foam precursors to take place. In the illustrated embodiment, device 700 can be folded into a more compact configuration. This configuration also allows the reactant container to come into contact with the heating elements, or to come into close enough proximity that the reactant chemicals can be heated to an appropriate pre-heat temperature.

In an embodiment, pre-heating storage unit 710 is receives power from a 112 volt power source. As such, unit 710 is able to receive power from a vehicle battery or other similar source. In alternate embodiments, unit 710 can be battery powered or powered through other sources.

In other embodiments, other types of pre-heating storage units are used in conjunction with the immobilizing device. For example, if the foam precursor package is not integrated with the device, a pre-heating storage unit can be a small, heatable chamber adapted to contain and heat the foam precursor packages separately from the main part of the device. Alternatively, the pre-heating storage unit can be configured to accept the entire device, in a folded or unfolded position. In still other embodiments, the foam precursors are not pre-heated prior to activation.

Referring again to FIG. 6, after the reactant chemicals are pre-heated, the device is obtained by the user, in block 604. In an embodiment, the user removes the device (or the precursor package) from the pre-heating storage unit. Then the user unfolds the device, if it is stored in a folded position. If the foam precursor package is not integrated with the device, the user inserts the package into the flexible bag, or otherwise attaches the precursor package to the flexible bag.

FIG. 8 illustrates a second perspective view of an immobilizing device 800, prior to activation, in a folded position, in accordance with an embodiment. In this embodiment, the base 802 of the device 800 includes grooves, indentations, or other mechanisms for easily folding the device 800 into a more compact configuration. Embodiment shown in FIG. 8 shows the device 800 being foldable so that the flexible bags 804 are located in outside, opposite facing positions, when the base 802 is folded. This facilitates heating the foam precursor packages, when the packages are integrated with the device. In other embodiments, the device 800 can be foldable in other configurations, or the base 802 can be relatively rigid, and not easily foldable.

FIG. 9 illustrates a perspective front view of an immobilizing device 900, prior to activation, in an unfolded position, in accordance with an embodiment. Referring again to FIG. 6, the user then accesses the back of the base, and exposes an adhesive surface at the back of the base, in block 606, by peeling away a protective sheet.

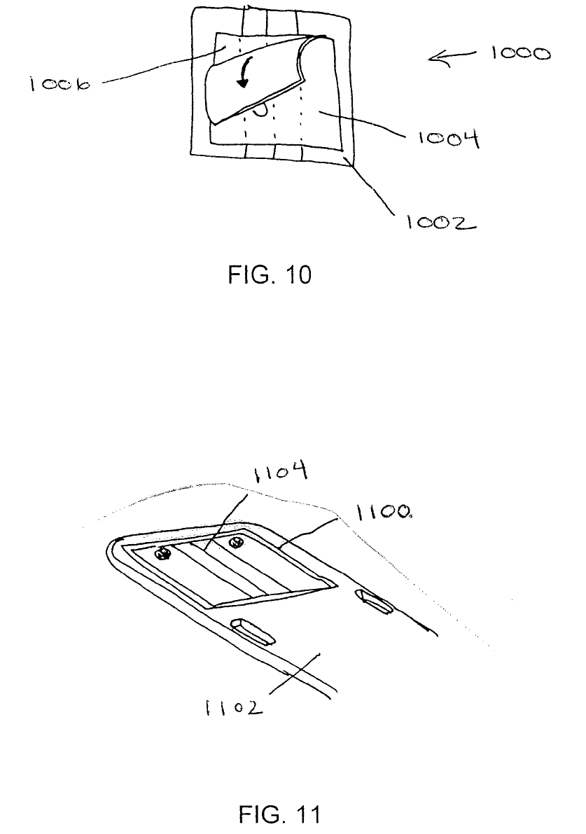

FIG. 10 illustrates a perspective back view of an immobilizing device 1000, prior to activation, with a portion of an adhesive protection sheet 1004 removed, in accordance with an embodiment. The adhesive protection sheet 1004 can be removed to expose an adhesive 1006 disposed on the back surface of the base 1002.

Referring again to FIG. 6, after the adhesive is exposed, the user affixes the device to a rigid or semi-rigid support structure, in block 608. This involves applying the adhesive on the back of the device to the appropriate surface of the support structure, and pressing down to ensure that the device is properly affixed to the surface of the structure. FIG. 11 illustrates a perspective view of an immobilizing device 1100, prior to activation, affixed to a rigid spine board 1102, in accordance with an embodiment. In an embodiment, the device 1100 includes a vertical guiding line 1104 printed on the surface, which facilitates proper alignment of the patient's head or other body part.

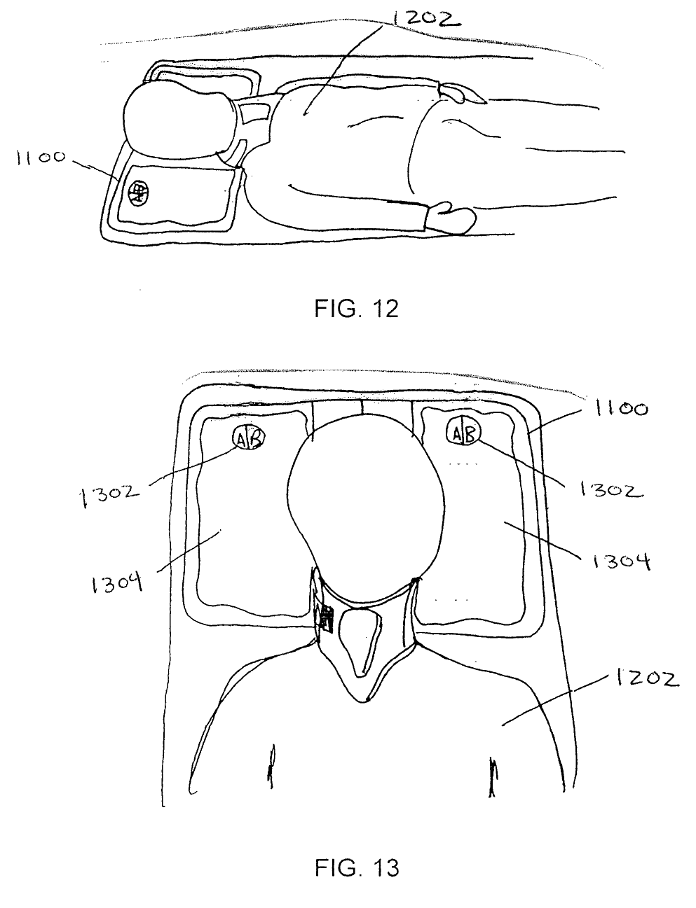

After affixing the device to the support structure, the portion of the patient to be immobilized is placed in proximity to the device, in block 610. FIG. 12 illustrates a perspective view of the immobilizing device of FIG. 11, prior to activation, with a patient 1202 positioned on the device 1100, in accordance with an embodiment. In the illustration, the patient is wearing a cervical collar to further stabilize his neck.

Once the patient is properly positioned, the foam precursor package or packages are activated, and the foam precursors are combined, in block 612, in order to react and fill the flexible bags with foam. In an embodiment, the foam precursors are combined by the user physically agitating (e.g., squeezing or pressing down) on a foam precursor package (e.g., package 400, FIG. 4) in a manner that a separation mechanism (e.g., mechanism 418, FIG. 4) of the package selectively breaks or deflects, allowing the foam precursors to combine and react. If the device includes more than one foam precursor package, the user can activate multiple packages at substantially the same time (e.g. by simultaneously agitating packages with the left hand and with the right hand), or the user can activate multiple packages in sequence.

FIG. 13 illustrates a perspective top view of the immobilizing device 1100 and patient 1202 of FIG. 12, prior to activation, in accordance with an embodiment. In an embodiment, the foam precursor packages 1302 are located within the flexible bags 1304 in a manner that they are easily accessible to the user, and their location is identified by markings on the surface of the flexible bags 1304. The user can press down on the packages 1302 until the user feels the packages selectively break or deflect, or until the user observes the formation of the foam. The foam fills and inflates the flexible bags, while causing the bags to contour around the back and/or sides of the patient's head (or other body part). The foam then hardens, thus at least partially or completely immobilizing the patient's head.

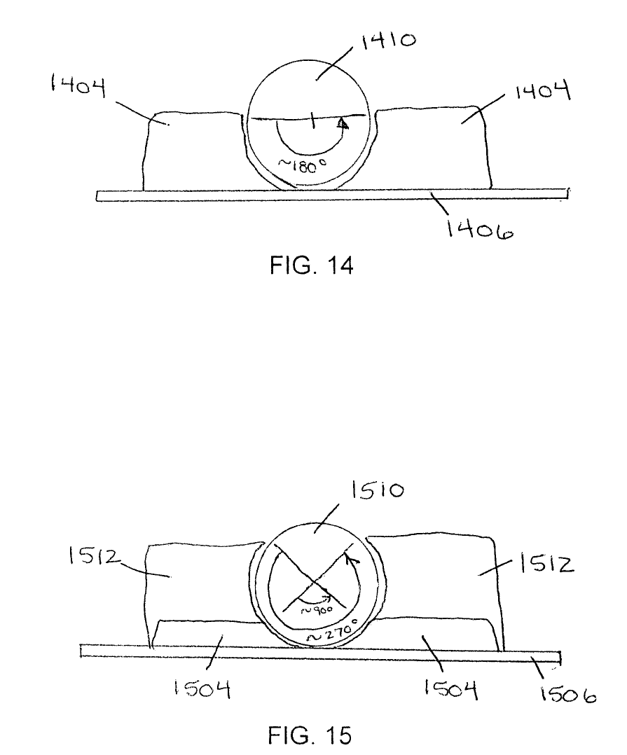

In an embodiment, the bags only partially contour around the patient's body part. This facilitates easy removal of the device from the body part from. FIGS. 14 and 15 illustrate cross-sectional, head-down views of an immobilizing device and body part. As illustrated in FIG. 14, in an embodiment, the bags 1404 fill and contour in a manner that support is provided by the bags 1404 and the base 1406 approximately 180 degrees around the body part 1410. Alternatively, as illustrated in FIG. 15, in another embodiment, support from the bags and base 1506 are provided in a range between 90 degrees and 270 degrees around the body part 1510. Specifically, bags 1504 illustrate an approximately 90 degree range of support, and bags 1512 (shown as superimposed over bags 1504) illustrate an approximately 270 degree range of support.

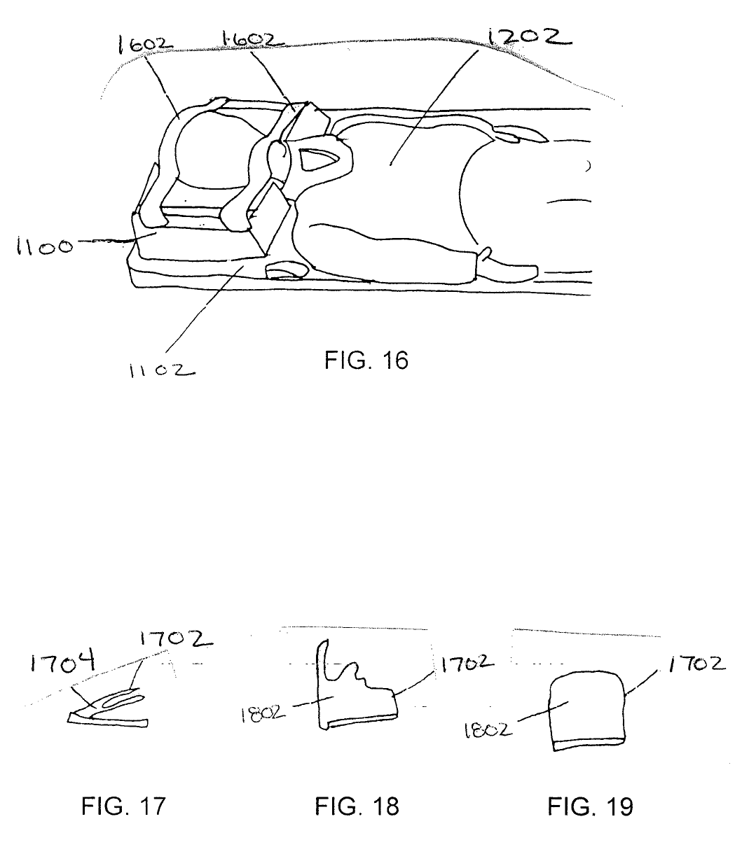

Referring again to FIG. 6, in block 614, the patient's head or other body part is more completely secured. FIG. 16 illustrates a perspective view of the immobilizing device 1100 and patient 1202 of FIG. 12, after activation, in accordance with an embodiment. As the figure illustrates, forehead and chin straps 1602 are attached to the device and/or the support structure 1102 in order to fully secure the patient. This results in a full cervical spine immobilization, in an embodiment, which is a basic protocol for a patient with a suspected head, neck or spinal cord injury.

The next figures illustrate inflation of the flexible bags during activation. FIG. 17 illustrates a side, cross-sectional view of a flexible bag 1702 of an immobilizing device, prior to activation, in accordance with an embodiment. In this pre-activation state, the flexible bag 1702 is deflated, and the interior cavity 1704 includes little or no air.

FIG. 18 illustrates a side, cross-sectional view of the flexible bag 1702 of FIG. 17, shortly after activation has occurred, in accordance with an embodiment. When the foam precursor package is activated, and the foam precursors are combined, foam 1802 begins to fill the interior cavity of the bag 1702. FIG. 18 illustrates the flexible bag 1702 in a partially filled state.

FIG. 19 illustrates a side, cross-sectional view of a flexible bag 1702 of FIG. 17, in a fully expanded position after activation, in accordance with an embodiment. In this state, the foam 1802 has fully inflated the bag. In areas where an interference exists (e.g., the surface of a patient's body), the bag and foam are contoured. Desirably, the foam rapidly hardens, providing a relatively firm support structure for the patient's body part.

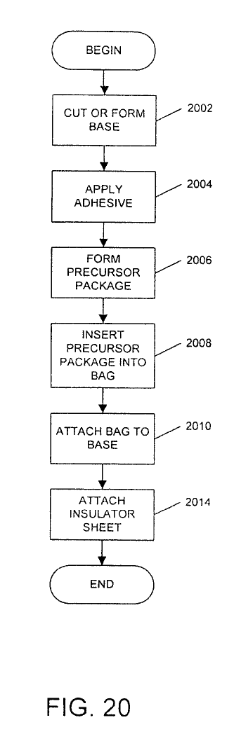

FIG. 20 illustrates a flowchart of a method for manufacturing an immobilizing device, in accordance with an embodiment. In block 2002, a base portion is cut or formed into an appropriate shape and size, depending on the size and shape of the intended body part (or other physical structure) that the device functions to immobilize. As described above, the base portion is cardboard or another rigid or semi-rigid material, in various embodiments. Alternatively, the base portion is a flexible or stiff material, such as a plastic sheet. Other suitable materials also can be used. In block 2004, an adhesive is applied to the back surface of the device, and a protective sheet is applied over the adhesive.

In an embodiment, in block 2006, the foam precursor package is formed, including the first and second foam precursor chambers. The chambers are filled with the appropriate foam precursor chemicals. In an embodiment, the foam precursor packages are inserted into and fixed within the flexible bags, in block 2008. As described previously, in other embodiments, the foam precursor packages are not integrated within the flexible bags, but instead are insertable into the bags or attachable to the bags.

The flexible bags are attached to the base portion, in block 2010. For example, the flexible bags are attached using an adhesive, in an embodiment. In other embodiments, the flexible bags are attached using another permanent, semi-permanent or other attachment mechanism.

The insulator sheets are attached to the base or to the flexible bags, in block 2012. The base is folded, in an embodiment, and the device is packaged for distribution. The method then ends.

In another embodiment, the device is attached to the support structure prior to distribution or use. In this embodiment, the base is attached to the support structure using an adhesive or other permanent, semi-permanent or other attachment mechanism. Alternatively the flexible bags can be attached directly to the support structure, using an adhesive or other permanent, semi-permanent or other attachment mechanism.

Various embodiments have been described of apparatus adapted to immobilize a portion of the body, along with descriptions of methods of using such apparatus, and methods for making such apparatus. Embodiments of the invention described above enable body parts, such as a patient's head, neck, spine and other parts to be immobilized and secured.

Other applications would be obvious, based on the description. For example, embodiments of the invention can be used to immobilize other portions of a patient's body, or other physical structures, for that matter. Further, embodiments of the invention can be used in conjunction with other support structures besides a rigid spine board.

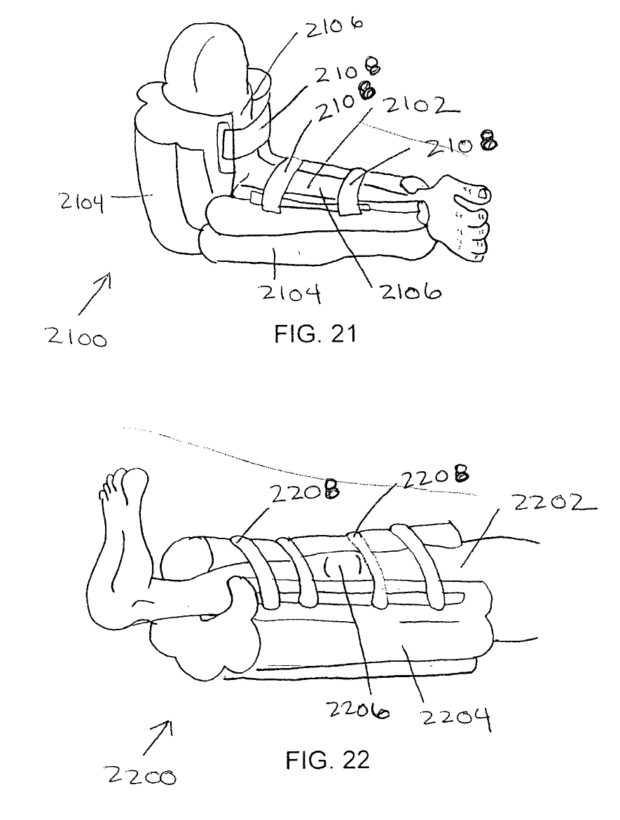

For example, a support structure and immobilizing device can be adapted from embodiments described herein to provide immobilization of a foot, knee, leg, torso, shoulder, arm, elbow, wrist, hand, finger, and/or virtually any other body part. For example, but not by way of limitation, FIG. 21 illustrates a perspective view of an immobilizing device 2100 with one or more flexible bags 2104 within which foam-producing chemicals are combined to produce a foam, which immobilizes a patient's arm 2102, in accordance with an embodiment. FIG. 22 illustrates a perspective view of an immobilizing device 2200 with one or more flexible bags 2204 within which foam-producing chemicals are combined to produce a foam, which immobilizes a patient's leg 2202, in accordance with another embodiment.

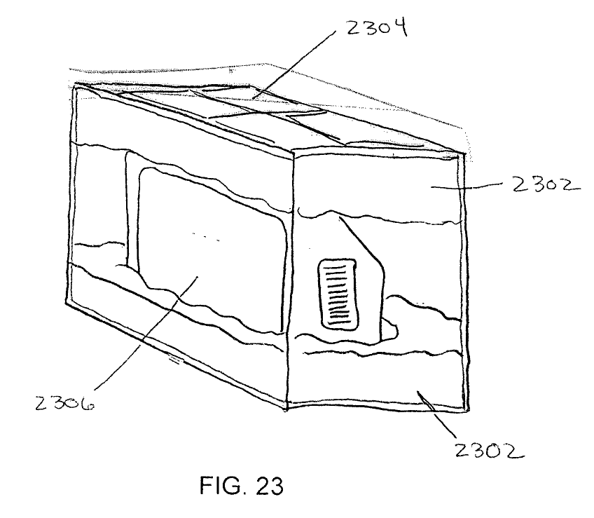

Besides body parts, embodiments of the invention also can be used to immobilize other types of physical structures. For example, but not by way of limitation, embodiments of the invention can be used to immobilize items within a shipping container. In such an embodiment, one or more immobilization devices, of various embodiments, can be placed in a shipping container in proximity to one or more objects being immobilized. Each immobilization device can be activated before or after being placed in the shipping container. FIG. 23 illustrates a perspective, cut-away view of two immobilization devices 2302 within a shipping container 2304, in accordance with an embodiment, where the immobilization devices 2302 are being used to immobilize a monitor 2306.

Although the support structure described herein is a substantially flat spine board, in other embodiments, other substantially flat support structures can be used, or support structures can be pre-contoured or contourable in a manner that they can be loosely contoured to the portion of the body or the other physical structure being immobilized. Other modifications, which would be apparent to those of skill in the art, could be made to various embodiments to achieve the same results.

While the foregoing examples of dimensions and ranges are considered typical, various embodiments of the invention are not limited to such dimensions or ranges. Various dimensions of the portions of the device and the support structure depend on the portion of the body or other structure being immobilized.

Although specific embodiments have been illustrated and described herein, it will be appreciated by those of ordinary skill in the art that any arrangement that is calculated to achieve the same purpose may be substituted for the specific embodiments shown. In addition, the arrangement of various steps of use and manufacture can be done in the order shown, or in a different order. Many adaptations of the invention will be apparent to those of ordinary skill in the art. Accordingly, this application is intended to cover any adaptations or variations of the invention. It is manifestly intended that this invention be limited only by the following claims and equivalents thereof.

1. An apparatus comprising:
a base;
at least one flexible bag, attached to the base;
one or more reactant chambers, which include a first mechanism that functions to hold at least two reactant chemicals separate, and second mechanism that functions to enable the reactant chemicals to combine; and
the at least two reactant chemicals, which function to produce a foam, when combined, which inflates an interior cavity of the flexible bag, and which causes the flexible bag to contour around a physical structure in proximity to an outside surface of the flexible bag.

2. The apparatus of claim 1, wherein the base is formed from a substantially rigid material.

3. The apparatus of claim 1, wherein the base is formed from a flexible material.

4. The apparatus of claim 1, wherein the one or more reactant chambers are attached to an inside surface of the at least one flexible bag.

5. The apparatus of claim 1, wherein the at least one flexible bag includes an opening to allow the foam to be introduced into the interior cavity.

6. The apparatus of claim 1, wherein the at least one flexible bag includes an opening to allow the at least two reactant chemicals to be introduced into the interior cavity.

7. The apparatus of claim 1, further comprising:
one or more regions of insulating material, located in proximity to the at least one flexible bag, wherein the one or more regions of insulating material function to insulate an object placed in proximity to the apparatus from heat produced by an exothermic reaction, which occurs when the at least two reactant chemicals are combined.

8. The apparatus of claim 1, further comprising:
a securing mechanism, which functions to secure an object placed in proximity to the apparatus.

9. An apparatus comprising:
a base;
a flexible bag, attached to the base, wherein the flexible bag has an inside surface that defines an interior cavity; and
a foam precursor package, which includes a first foam precursor and a second foam precursor, wherein the first foam precursor and the second foam precursor, when combined, function to produce a reaction that results in formation of a foam, and wherein the flexible bag functions to substantially contain the foam within the interior cavity during the reaction.

10. The apparatus of claim 9, wherein the foam precursor package further comprises:
a first chamber, within which the first foam precursor is disposed prior to the reaction;
a second chamber, within which the second foam precursor is disposed prior to the reaction; and
at least one separation mechanism which, when agitated, functions to enable the first foam precursor and the second foam precursor to combine and produce the reaction.

11. The apparatus of claim 10, wherein the at least one separation mechanism includes at least one frangible barrier which breaks when agitated.

12. The apparatus of claim 9, wherein the flexible bag further includes a sealable opening having a size sufficient to accept insertion of the foam precursor package into the interior cavity.

13. The apparatus of claim 9, wherein the foam precursor package is attached to the inside surface of the flexible bag within the interior cavity.

14. A method for immobilizing an object, the method comprising:
positioning the object in proximity to an immobilizing device, which includes
a base;
at least one flexible bag attached to the base;
one or more reactant chambers, which include a first mechanism that functions to hold at least two reactant chemicals separate, and second mechanism that functions to enable the reactant chemicals to combine;
the at least two reactant chemicals, which function to produce a foam, when combined, which inflates an interior cavity of the flexible bag, and which causes the flexible bag to contour around the object; and
combining the reactant chemicals by physically agitating the reactant chamber, which results in the reactant chemicals producing a foam that inflates the flexible bag and causes the flexible bag to contour around the object.

15. A method for manufacturing an immobilizing apparatus, the method comprising:
forming a base portion, having a front surface and a back surface;
attaching a flexible bag to the front surface of the base portion, wherein the flexible bag includes an interior cavity; and
forming at least one reactant chamber having a first chamber and a second chamber, wherein the first chamber includes a first reactant chemical, and the second chamber includes a second reactant chemical which, when combined with the first reactant chemical, functions to produce a foam, which substantially fills the interior cavity of the flexible bag.

16. The method of claim 15, further comprising:
inserting the at least one reactant chamber into the interior cavity of the flexible bag; and
attaching the at least one reactant chamber within the interior cavity of the flexible bag.

17. The method of claim 15, further comprising:
attaching one or more regions of insulating material to the apparatus in proximity to the at least one flexible bag, wherein the one or more regions of insulating material function to insulate an object placed in proximity to the apparatus from heat produced by an exothermic reaction, which occurs when the first reactant chemical and the second reactant chemical are combined.

18. The method of claim 15, further comprising:
applying an adhesive to the back surface of the base portion.

19. A splint for at least partially immobilizing a body part, the splint comprising:
a flexible bag having an inside surface that defines an interior cavity, wherein the flexible bag is shaped to at least partially contour to a body part intended to be at least partially immobilized; and
a foam precursor package, which includes a first foam precursor and a second foam precursor, wherein the first foam precursor and the second foam precursor, when combined, function to produce a reaction that results in formation of a foam, and wherein the flexible bag functions to substantially contain the foam within the interior cavity during the reaction and to contour at least partially around the body part.

20. The splint of claim 19, further comprising:
a base to which the at least one flexible bag is attachable.

21. The splint of claim 19, wherein:
the first foam precursor includes a member of the isocyanate family with a functionality in a range of 2.0-2.9; and
the second foam precursor includes a polyol, and wherein the first foam precursor and the second foam precursor, when combined, function to produce the reaction that results in formation of a polyurethane foam.

22. The splint of claim 21, wherein the second foam precursor further includes one or more additives selected from a group of additives that includes a blowing agent precursor, a surfactant, a catalyst, a cross-linker, and a flame retardant.
